(19) United States
(12) Patent Application Publication
(10) Pub. No.: US 2026/0103581 A1
(43) Pub. Date: Apr. 16, 2026

(54) PROCESS FOR GRANULATING AZO COMPOUNDS AND GRANULES OBTAINED

(71) Applicant: ARKEMA FRANCE, COLOMBES (FR)

(72) Inventors: Jean-Marc SAGE, Pierre-Benite (FR); Jean-Michel BOSSOUTROT, Pierre-Benite (FR); Chloé GONNET, Pierre-Benite (FR)

(73) Assignee: ARKEMA FRANCE, COLOMBES (FR)

(21) Appl. No.: 19/117,216

(22) PCT Filed: Oct. 2, 2023
(86) PCT No.: PCT/FR2023/051522

§ 371 Date: Mar. 31, 2025

(30) Foreign Application Priority Data
Oct. 3, 2022 (FR) 2210085

(51) Int. Cl. C08K 5/23; A61K 9/16; C08K 7/16

(52) U.S. Cl. CPC C08K 5/23; A61K 9/1682; C08K 7/16

(57) ABSTRACT

The present invention relates to a process for granulating an azo compound, comprising the following steps:
a) a granulation step by stirring an aqueous suspension of said azo compound, in the presence of an organic binder;
b) an optional step of recovering the granules obtained in step a), preferably by filtration; and
c) an optional step of drying the granules recovered in step b).

It also relates to granules obtainable by the process according to the invention and to uses thereof.

The present invention relates to a process for granulating azo compounds, and to the granules obtainable by this process and the uses thereof.

Azo compounds, and more particularly 2,2′-azobis(isobutyronitrile) called AZDN or AIBN, are well-known products. They are used as swelling agents, synthesis intermediates or initiators of polymerization reactions using free radicals. These reactions can be polymerization reactions in bulk, in solution, in suspension or in emulsion and can employ very varied monomers, for example (meth)acrylic or vinyl monomers, such as acrylamide, acrylonitrile, alkyl (meth)acrylate, styrene, vinyl chloride and acetate or vinylidene chloride. The fields of application are therefore very diverse and concern in particular (but not exclusively) acrylic sheets or fibres, flocculants, paints, coating resins, grafted polyols, polystyrene, PVC, PVA, or else PMMA.

These azo compounds are conventionally obtained by oxidizing the hydrazo derivatives or the corresponding amino nitriles as described for example in documents U.S. Pat. No. 2,515,628, WO 03/002521, U.S. Pat. No. 3,390,146, CN 1309705 or else EP 2 821 393. After oxidation, the resulting suspension is generally drained, then optionally dried, to give a solid in powder form. However, azo compounds in powder form pose many problems.

In particular, they generate dusts likely to present a risk of explosion and/or an industrial hygiene risk. These dusts, indeed, can come into contact with the upper respiratory tract of people who handle them, after they have been accidentally suspended in the air. Such contact may occur with operators present at the industrial facility, for example when manually loading the powder into the reactors, or with operators who take samples necessary for control of the manufacturing process. It is desirable to limit to a minimum, or even completely avoid, any contact of this type, in order to ensure the safety of operators and to guarantee the hygiene of manufacturing premises.

In addition, this type of powder is not easily handled by operators. The powder is generally not flowable enough to lend itself to easy transportation and loading into the reactors. Azo compounds in powder form have poor flowability, often leading to caking problems during their storage and loading.

There is therefore a need for improved shaping of azo compounds, in particular which generates little or no dust and which is easily handled by operators.

Thus one objective of the present invention is to provide a granulation process which is easy to implement, and preferably more environmentally friendly.

Another objective of the present invention is to provide granules of azo compounds which avoid or reduce dust and/or which can be transported and handled easily.

An objective of the present invention is to provide granules of azo compounds which are solid (that is to say resistant), in particular which retain their shape during their storage and their handling.

Another objective of the present invention is to provide granules of azo compounds having properties similar to those of powders.

The present invention meets all or part of the above objectives.

The inventors have found that the granulation of azo compounds can be carried out by means of a process using an aqueous suspension of these compounds, with stirring and in the presence of an organic binder. This process makes it possible in particular to avoid using organic solvents in large quantities, the suspension being aqueous. It can also make it possible to obtain granules without using surfactants or dispersants.

For example, mechanical-type granulation is known from document WO 00/24706. This involves compressing then extruding powders of azo compounds to make granules. However, it is essential for this technique to control the shaping temperature. However, such control is not obvious at the industrial level. This type of process involves risks of heating by friction or compression and consequently risks of violent thermal decomposition of the azo compounds during the formation of the granules. In addition, the granules obtained by mechanical granulation are not very resistant to crushing and disintegrate easily.

The process according to the invention avoids these risks of heating and decomposition by using gentle granulation conditions, which makes it possible to obtain granules of high purity. The application properties of the compounds, particularly in polymerization, are thus preserved.

In addition, and surprisingly, the granules according to the invention are solid (resistant). More particularly, they have a crushing resistance that is significantly greater than other known shaped forms of azo compounds.

In particular, “crushing resistance” means the maximum weight per unit of a surface composed of granules that these granules can support before being crushed or disintegrating (i.e. losing their shape):

Crushing resistance=load weight at breaking point/granule surface area

To measure crushing resistance, generally, a measurement is made of the compressive load necessary to cause the granule to break.

The crushing resistance of the granules according to the invention is in particular greater than or equal to 25 g/cm², more preferably greater than or equal to 40 g/cm².

They preferably have a maximum crushing resistance of 90 g/cm², preferably 95 g/cm², more preferably 100 g/cm² or even 500 g/cm², endpoints included. For example, their maximum crushing resistance is between 50 and 200 g/cm², preferably between 55 and 95 g/cm². They can therefore be transported and handled without breaking or disintegrating.

Being preferably substantially spherical, they can flow easily from drums or storage bags and loading the reactors is facilitated.

Thus, the granules according to the invention are preferably substantially spherical, or even spherical, and can have a diameter of between 0.5 mm and 5 mm, preferably between 2 and 3 mm. They are therefore generally larger in size than dusts and in particular much larger in size than inhalable dusts. In particular, “dust” means particles less than 100 μm in size. More particularly, inhalable dusts are less than 20 μm, preferably less than 5 μm in size. Also surprisingly, the granules obtained have properties similar to those of powders and in particular a similar dissolution time. The granules according to the invention are therefore entirely suitable for industrial use, like the usual powders.

### BRIEF DESCRIPTION OF THE INVENTION

The present invention relates to a process for granulating an azo compound of general formula (I) below:

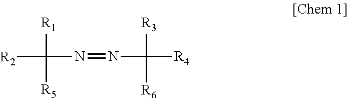

in which:
the radicals R¹, R², R³ and R⁴, identical or different, are chosen independently of one another from:
   a linear or branched alkyl group, preferably a (C₁-C₆)alkyl group, optionally substituted by a hydroxy or alkoxy group or by a halogen atom;
   a cycloalkyl group, preferably a (C₃-C₆) cycloalkyl group, optionally substituted by a hydroxy or alkoxy group or by a halogen atom;
   an aryl group, preferably phenyl or naphthyl, optionally substituted by a hydroxy, alkyl or alkoxy group or by a halogen atom;
   an aralkyl group, preferably benzyl or phenethyl, optionally substituted by one or more alkyl, alkoxy or hydroxy group(s) or by one or more halogen atom(s); or
   at least one of the combinations of R¹ with R² and/or of R³ with R⁴ forms, with the carbon atom to which it is (or they are) connected, a cycloalkyl radical or a C(O) group;
the radicals R⁵ and R⁶, identical or different, are chosen independently of one another from the CN or NH₂ groups;
   said process comprising the following steps:
a) a granulation step by stirring an aqueous suspension of said azo compound, in the presence of an organic binder;
b) an optional step of recovering the granules obtained in step a), preferably by filtration; and
c) an optional step of drying the granules recovered in step b).

The present invention also relates to granules obtainable or obtained or directly obtained by the process such as according to the invention.

The present invention also relates to granules comprising an azo compound of general formula (I) and an organic binder, as defined below.

The present invention also relates to granules having a crushing resistance greater than or equal to 25 g/cm², more preferably greater than or equal to 40 g/cm².

The present invention relates to the use of such granules as swelling agents, initiators of polymerization reactions using free radicals, or else as synthesis intermediates, particularly in the preparation of pharmaceutical or agrochemical compounds.

### DETAILED DESCRIPTION OF THE INVENTION

The azo compounds have the following general formula (I):

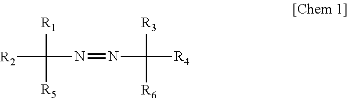

in which:
the radicals R¹, R², R³ and R⁴, identical or different, are chosen independently of one another from:
   a linear or branched alkyl group, preferably a (C₁-C₆)alkyl group, optionally substituted by a hydroxy or alkoxy group or by a halogen atom;
   a cycloalkyl group, preferably a (C₃-C₆) cycloalkyl group, optionally substituted by a hydroxy or alkoxy group or by a halogen atom;
   an aryl group, preferably phenyl or naphthyl, optionally substituted by a hydroxy, alkyl or alkoxy group or by a halogen atom;
   an aralkyl group, preferably benzyl or phenethyl, optionally substituted by one or more alkyl, alkoxy or hydroxy group(s) or by one or more halogen atom(s); or
   at least one of the combinations of R¹ with R² and/or of R³ with R⁴ forms, with the carbon atom to which it is (or they are) connected, a cycloalkyl radical (preferably a (C₃-C₆) cycloalkyl) or a C(O) group;
the radicals R⁵ and R⁶, identical or different, are chosen independently of one another from the CN (nitrile) or NH₂ groups.

Preferably, R⁵ and R⁶ are identical.

In particular, when R⁵ and R⁶ are identical and represent an NH₂ group, R¹ with R², and R³ with R⁴, each form, with the carbon atom to which they are connected, a C(O) group; or when R⁵ and R⁶ are identical and represent a CN group, the radicals R¹, R², R³ and R⁴, identical or different, are chosen independently of one another from:
a linear or branched alkyl group, preferably a (C₁-C₆)alkyl group, optionally substituted by a hydroxy or alkoxy group or by a halogen atom;
a cycloalkyl group, preferably a (C₃-C₆) cycloalkyl group, optionally substituted by a hydroxy group or by a halogen atom;
an aryl group, preferably phenyl or naphthyl, optionally substituted by a hydroxy, alkyl or alkoxy group or by a halogen atom;
an aralkyl group, preferably benzyl or phenethyl, optionally substituted by one or more alkyl, alkoxy or hydroxy groups or by one or more halogen atoms; or
at least one of the combinations of R¹ with R² and/or of R³ with R⁴ forms, with the carbon atom to which it is (or they are) connected, a cycloalkyl radical.

Most preferably, R⁵ and R⁶ represent a CN (nitrile) group. In particular, R¹ and R³ are identical and/or R² and R⁴ are identical.

Preferably, the radicals R¹, R², R³ and R⁴ are chosen from methyl, ethyl, n-propyl, isopropyl, n-butyl, isobutyl, sec-butyl, tert-butyl, cyclopropyl and phenyl.

In particular, the radicals R¹, R², R³ and R⁴ are chosen from alkyl groups as defined above. In particular, when the substituent(s) are alkyls and/or alkoxys, they comprise between 1 and 6 carbon atoms. The halogens include in particular fluorine, chlorine, bromine and iodine.

Preferably, the compounds of general formula (I) are symmetrical.

Examples of azo compounds of general formula (I) include:
2,2′-azobis(isobutyronitrile),
2,2′-azobis(2,4-dimethylvaleronitrile),
2,2′-azobis(2-methylhexylonitrile),
2,2′-azobis(2-cyclopropylpropionitrile),
2,2′-azobis(2-phenylpropionitrile),
2,2′-azobis(2-methylbutyronitrile),
1, 1′-azobis(1-cyclohexanecarbonitrile), and azodicarbonamide.

The corresponding structures are given in the table below:

TABLE 1

| Name | Structure |
|---|---|
| 2,2′-azobis(isobutyronitrile) | 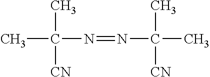 |
| 2,2′-azobis(2,4-dimethylvaleronitrile) | 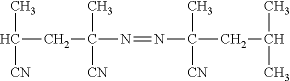 [SMILES: CC(C)CC(C)(C#N)N=NC(C)(C#N)CC(C)C#N] |
| 2,2′-azobis(2-methylhexylonitrile) | 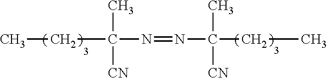 |
| 2,2′-azobis(2-cyclopropylpropionitrile) | 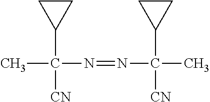 [SMILES: CC(C#N)(N=NC(C)(C#N)C1CC1)C1CC1] |
| 2,2′-azobis(2-phenylpropionitrile) | 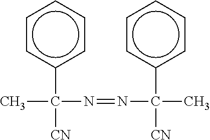 |
| 2,2′-azobis(2-methylbutyronitrile) | 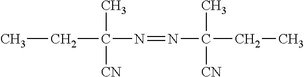 |
| 1,1′-azobis(1-cyclohexanecarbonitrile) | 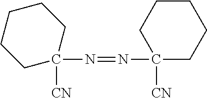 [SMILES: N#CC1(N=NC2(C#N)CCCCC2)CCCCC1] |
| azodicarbonamide | 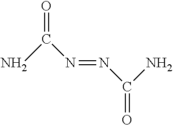 |

The preferred compound according to the invention is 2,2′-azobis(isobutyronitrile), generally called AZDN or AIBN. It corresponds to the compound for which R¹, R², R³ and R⁴ are a methyl group and R⁵ and R⁶ are a CN group (CAS No. 78-67-1).

Before its granulation, the azo compound of formula (I) is generally in the solid state, most often in powder form. Thus, said azo compound is preferably in the form of a powder comprising particles having a size of between 10 μm and 200 μm, preferably between 40 μm and 150 μm. In particular, said powder has a particle size distribution (Dv50) of between 10 μm and 200 μm, preferably between 40 μm and 150 μm, more preferably between 90 μm and 130 μm.

The particle size of a powder is generally defined as the statistical distribution of the particles that make up the powder according to their dimensions (size and shape of elementary particles). The particle size distribution (Dv50) is a known parameter. It corresponds to the particle diameter (μm) below which 50% of the particle volume is located on the distribution curve expressed as cumulative frequency. For example, if Dv50=100 μm, 50% of the sample particle volume has a diameter less than 100 μm and 50% of the particle volume has a diameter greater than 100 μm. The two other characteristic diameters usually used to describe the particle size distribution of powders are Dv10 and Dv90:
Dv10: it corresponds to the diameter (μm) below which 10% of the particle volume is located; and
Dv90: it corresponds to the diameter (μm) below which 90% of the particle volume is located.

In particular, AZDN (especially dry) is in the form of a powder with a Dv50 of between 80 μm and 200 μm, preferably between 90 μm and 110 μm.

In particular, AZDN (especially wet) is in the form of a powder with a Dv50 of between 80 μm and 200 μm, preferably between 110 μm and 130 μm.

Said azo compound powder can thus comprise between 0.1% and 10%, preferably between 5% and 10% by weight of water, relative to the total weight of the powder.

Preferably, said compound of formula (I) has a solubility in the organic binder of at least 10 g/L, preferably between 10 g/L and 100 g/L and more preferably between 20 g/L and 70 g/L.

Said azo compound is therefore preferentially soluble in said organic binder.

This solubility can be determined using standard methods. It can also be determined as follows:

1.5 g of compound of formula (I) are added to 10 g of organic binder at 20° C. The bottle is shaken for a period of between 1 h and 10 h, for example 6 h. After decantation, said binder is analysed to determine its content of compound of formula (I): this content corresponds to the solubility of the azo compound in the organic binder in g/L. The analysis is carried out by gas chromatography. A standard range is produced between 0 and 150 g/l by diluting the compound of formula (I) in acetone.

The azo compounds of formula (I) according to the invention are known and are available commercially, generally in the form of a powder.

One can for example cite AZDN marketed by the company Arkema, the products V-40, V-59, AIBN, V-65 marketed by Fujifilm Wako or the products Vazo®52, Vazo@64, Vazo®67 and Vazo®88 marketed by Chemours.

Use can also be made of an azo compound recovered directly after its production. For example, it is possible to use the azo compound drained, washed and obtained in the form of a wet powder after oxidation of the corresponding hydrazo or amino-nitrile compound. In this case, the powder can comprise between 5% and 10% by weight of water, relative to the total weight of the powder.

This same azo compound can be used, drained, washed and then dried. In this case, the powder can comprise between 0.01% and 0.2% by weight of water, relative to the total weight of the powder.

The granulation step a) for the azo compound is carried out in the presence of an organic binder. Said organic binder is preferably liquid (under the operating conditions of the process according to the invention).

In particular, said organic binder is poorly soluble in water. By “poorly soluble in water” is meant in particular a binder which has a solubility in water of less than or equal to 25 g/L, preferably between 0.1 g/L and 20 g/L, more preferably between 5 g/L and 15 g/L.

To measure the solubility of the organic binder in water, conventional methods can be used.

One can also mix 100 g of water and 20 g of binder with stirring at 20° C. for 1 h. The aqueous phase is decanted and then analysed by gas chromatography in order to determine the binder content in g/L, corresponding to its solubility.

Preferably, the organic binder is chosen from aliphatic acetates, aliphatic carbonates, non-halogenated aromatic hydrocarbons, aliphatic ketones and aliphatic ethers, or mixtures thereof.

Among the aliphatic acetates, alkyl acetates are preferred, said alkyl being able to comprise at least 4 carbon atoms, preferably between 4 and 6 carbon atoms. They have in particular the following formula:

CH₃—C(O)O—Rₐ, Rₐ being an alkyl comprising at least 4 carbon atoms, preferably between 4 and 6 carbon atoms.

Among the aliphatic carbonates, dialkyl carbonates are preferred, said alkyls being able to comprise at least 2 carbon atoms, preferably between 2 and 3 carbon atoms. They have in particular the following formula:
R_{b}—O—C(O)—O—R_{c}, R_{b} and R_{c} being independently of one another an alkyl comprising at least 2 carbon atoms, preferably between 2 and 3 carbon atoms. Preferably R_{b} and R_{c} are identical. By non-halogenated aromatic hydrocarbons are meant in particular alkylbenzenes and preferably toluene, xylene (o-, m- and p-xylenes) and cumene. By alkylbenzene is meant in particular a benzene substituted by at least one alkyl comprising at least 1 carbon atom, preferably between 1 and 5 carbon atoms, more preferably methyl.

Among the aliphatic ketones, dialkyl ketones are preferred, which may comprise at least 6 carbon atoms, preferably between 6 and 8 carbon atoms. They have in particular the following formula: CH₃—C(O)—R_{d}, Rₐ being an alkyl comprising at least 4 carbon atoms, preferably between 4 and 6 carbon atoms.

Among the aliphatic ethers, dialkyl ethers are preferred, which may comprise at least 6 carbon atoms, preferably between 6 and 8 carbon atoms. They have in particular the following formula: CH₃—O—R_{f}, R_{f} being an alkyl comprising at least 5 carbon atoms, preferably between 5 and 7 carbon atoms, and preferably cyclic.

It is understood that said alkyls mentioned above may be linear, branched or cyclic.

Preferably, the organic binder is chosen from the group consisting of:
toluene, m-xylene, p-xylene, o-xylene, n-butyl acetate, isobutyl acetate, cyclopentyl methyl ether (CPME or methoxycyclopentane), diethyl carbonate, methyl isobutyl ketone and methyl pentyl ketone.

Preferably, the organic binder is chosen from the group consisting of:
toluene, m-xylene, n-butyl acetate, isobutyl acetate, cyclopentyl methyl ether (CPME or methoxycyclopentane) and methyl isobutyl ketone.

The preferred organic binders are isobutyl acetate and cyclopentyl methyl ether, more preferably isobutyl acetate.

Before step a) of granulation, the process may comprise a step of preparing the aqueous suspension of the azo compound (i.e. the heterogeneous mixture in which the liquid phase is water and the solid dispersed phase is the azo compound). For example, the azo compound is mixed in the form of a powder, preferably as defined above, with water, and preferably with stirring. Those skilled in the art can prepare this suspension by any conventional method.

In the context of the present invention, it is also possible to use the acidic aqueous suspension of azo compound obtained directly after the step of oxidation of the hydrazo compound or of the corresponding amino-nitrile (for example, after the step of chlorination of hydrazo-bis-isobutyronitrile, in the case of AZDN). Such a suspension may comprise between 2% and 15% by weight of HCl, relative to the total weight of the suspension.

In said suspension, the azo compound/water mass ratio can be between 1/99 and 40/60, preferably between 10/90 and 25/75; and more preferably between 15/85 to 25/75.

The process according to the invention comprises a step a) of granulating an azo compound of general formula (I) as defined above, suspended in water, by stirring, and in the presence of an organic binder.

Preferably, the organic binder is added to the aqueous suspension. It is possible to add the organic binder to the azo compound, then add the water, or to put all three components at the same time in the reactor, although these embodiments are not preferred.

The addition of the organic binder can be made by any means known to those skilled in the art. It can be one-off or continuous, preferably one-off. Indeed, it is not necessary to add the organic binder gradually: the total quantity of the latter can be added in one go to the aqueous suspension of azo compound. It is thus preferred to carry out a rapid addition of the organic binder, for example for a duration of between 1 min and 30 min, more preferably between 1 min and 20 min, in particular between 1 min and 5 min.

Granulation step a) is carried out with stirring. This stirring can be carried out by any known stirring means or element, for example any type of blade (straight or inclined) or helical ribbon.

The reactor may include one or more stages of stirring element(s).

More particularly, the stirring speed (corresponding for example to the rotation speed of the stirring element) must be sufficient to obtain a homogeneous suspension of the azo compound in water and a homogeneous dispersion of the organic binder, but without causing an emulsion to form. A speed of 500 to 900 rpm is generally used when operating in a reactor of 1 to 10 litre(s) or a speed of 50 to 300 rpm can be used when operating in a reactor of the order of 100 litres. For example, it is possible to increase or decrease the stirring speed throughout step a), though it is preferred to decrease it gradually.

Generally speaking, the formation of granules occurs quickly, i.e. a few minutes after stirring in the presence of said organic binder. In particular, the granules are formed over a period ranging from 1 min to 30 min, for example between 2 min and 10 min. Spherical (or substantially spherical) granules having a maximum diameter of between approximately 0.5 and 5 mm, for example between 1 and 5 mm, preferably between 2 and 3 mm, are generally obtained. Maintaining stirring after their formation can serve to consolidate the granules obtained and/or to reduce their dispersion.

Step a) can be carried out for a period of between 1 minute and 10 h, preferably between 30 min and 5 h, more preferably between 1 h and 5 h.

Granulation step a) may comprise or consist of the following two steps:
A1) addition of the organic binder to the aqueous suspension of azo compound to obtain a granulation medium, with stirring; then
A2) maintaining stirring of the granulation medium.

Preferably, step a) is carried out in the absence of surfactants and/or dispersing agents.

Preferably, step a) is carried out without adding surfactants and/or dispersing agents.

For example, step a) is carried out in the absence or without addition of sodium dioctylsulfosuccinate.

Granulation step a) is in particular carried out at a temperature which does not cause degradation of the azo compound. It can be between 5° C. and 45° C., preferably between 10° C. and 40° C., for example between 10° C. and 20° C. It is generally carried out at atmospheric pressure.

The azo compound/water mass ratio can be between 1/99 and 40/60, preferably between 10/90 and 25/75; and more preferably between 15/85 and 25/75.

The organic binder/azo compound mass ratio can be between 0.2 and 0.5, preferably between 0.3 and 0.5, more preferably between 0.3 and 0.4.

The process according to the invention optionally comprises subsequent steps of recovery and drying of the granules obtained in step a). These steps can be carried out in a conventional manner. For example, the granules can be recovered by filtration. They can then be dried. In particular, they can be dried at a temperature of between 10° C. and 45° C., preferably between 20° C. and 40° C. They can be dried under reduced pressure or more preferably with flushing with air, depleted air or an inert gas such as nitrogen. Drying can last a few hours, for example between 1 and 10 h, preferably between 3 and 5 h.

By “granules” are meant, in particular, solid and cohesive agglomerates of constituent particles, said particles being able to have a size of between 10 μm and 200 μm, preferably between 90 μm and 110 μm.

Thus, the present invention relates to granules obtainable (or obtained or directly obtained) by the process according to the invention. Such granules are new.

The present invention also relates to granules comprising an azo compound of general formula (I) as defined above and an organic binder as defined above.

In particular, in the granules according to the invention, said binder is present in trace amounts; particularly when these are dried after recovery, which leads to evaporation of the organic binder. The granules according to the invention can thus comprise between 20 ppm and 3000 ppm, for example between 20 ppm and 1000 ppm of organic binder. More particularly, they comprise between 20 ppm and 500 ppm of organic binder, more preferably between 50 ppm and 300 ppm of organic binder, more preferably still between 50 ppm and 200 ppm of organic binder, particularly after drying.

In particular, the granules according to the invention have a crushing resistance of greater than or equal to 25 g/cm², more preferably greater than or equal to 40 g/cm².

They advantageously have a maximum crushing resistance of 90 g/cm², preferably 95 g/cm², more preferably 100 g/cm² or even 500 g/cm², endpoints included. For example, their maximum crushing resistance is between 50 and 100 g/cm², preferably between 55 and 95 g/cm².

The granules according to the invention are generally of substantially spherical or spherical shape. They can have a diameter of between 0.5 and 5 mm and more preferably between 1 and 5 mm, for example between 2 and 3 mm.

The present invention also relates to granules comprising an azo compound of general formula (I) as defined above and having a crushing resistance of greater than or equal to 25 g/cm², more preferably greater than or equal to 40 g/cm². Such granules may include one or more of the characteristics mentioned above.

One of the advantages of granules such as according to the invention is their solidity, which allows them to be transported and handled very easily. They are also of high purity, with a low content of residual organic binder.

The present invention relates to their use as swelling agents, initiators of polymerization reactions using free radicals, or else as synthesis intermediates, particularly in the preparation of pharmaceutical or agrochemical compounds. The polymerization reactions can be bulk, solution, suspension or emulsion polymerization reactions and can employ very varied monomers, for example (meth)acrylic or vinyl monomers, such as acrylamide, acrylonitrile, alkyl (meth)acrylate, styrene, vinyl chloride and acetate or vinylidene chloride. The areas of application concern in particular (but not exclusively) acrylic sheets or fibres, flocculants, paints, coating resins, grafted polyols, polystyrene, PVC (poly(vinyl chloride)), PVA (polyvinyl acetate), or else PMMA (polymethyl methacrylate).

In particular, the granules according to the invention can be used in the preparation of polyols grafted with a mixture of styrene and acrylonitrile or else in the preparation of polyacrylonitriles as precursors of carbon fibres.

### EXAMPLES

The particle size of the dry AZDN used in the examples is 52.5/101/180 microns respectively dv(10), dv(50) and dv(90). That of the wet AZDN is 61/119/203 microns respectively dv(10), dv(50) and dV (90).

This particle size measurement is carried out using a Masterziser® S device. The measurement is made using water and a drop of Igepal® surfactant (ethoxylated nonylphenol) as a dispersant. The particle size measurement is carried out after 10 minutes of circulation in the measuring cell.

### Example 1: Granulation in a 500 ml Reactor

### 1-Operating Modes:

### Granulation:

The reactor is a 500 ml double-walled glass reactor, maintained at 15° C. by circulating cold water. It is equipped with a mechanical stirrer.

22.4 g of dry AZDN are weighed into a beaker, then 100 ml of water are added. After having homogenized the mixture using a spatula, the aqueous suspension is transferred into the reactor. 100 ml of water are then added to recover the AZDN remaining in the beaker.

The suspension, in the reactor, is stirred at 1000-1100 rpm, so that the AZDN remaining on the surface is entrained by the stirring. After a few minutes, the organic binder is quickly added to the reactor. The stirring speed is then reduced to 850 rpm.

After approximately three hours of stirring, the formation or otherwise of granules is recorded.

The reactor is drained onto a filter. Optionally, the aqueous filtration solution, saturated with binder, is used to finish rinsing the reactor.

The filtered granules are washed and allowed to dry in the open air under ventilation for approximately 24 h. The fragility of the formed and dried granules is recorded via their resistance to manual crushing.

### Measurement of Solubility of Organic Binder in Water:

The measurements were carried out by bringing 100 g of water and 20 g of organic binder into contact with stirring at 20° C. for 1 h. The aqueous phase is decanted and then analysed by gas chromatography to determine the concentration of organic binder.

### Measurement of the Solubility of AZDN in the Organic Binder:

The measurements were carried out by adding 1.5 g of AZDN to 10 g of organic binder at 20° C. The bottle is shaken for approximately 6 h. After decantation, the organic binder is analysed to determine its AZDN concentration. The analysis is carried out by gas chromatography with an injector temperature set at 220° C. (under these conditions, AZDN is essentially transformed into tetramethylsuccinonitrile in the injector). A standard range is produced between 0 and 150 g/l by diluting AZDN in acetone.

### Gas Chromatographic Analyses:

The chromatographic column is an OV1701 macrobore column (diameter=0.25 mm, length=30 m, film thickness=0.25 microns), the chromatographic device is a Hewlett Packard HP 6890 device equipped with an FID (flame ionization) detector.

### 2-Results Obtained:

The results obtained are presented in the following table:

TABLE 2

| | | Binder | AZDN | | | Size of | |
|---|---|---|---|---|---|---|---|
| | | solubility | solubility | | Formation | granules | *Solidity |
| | | in water | in water | Binder/AZDN | of | obtained | after |
| Test | Binder | (g/l) | (g/l) | mass ratio | granules | (mm) | drying |
| A | meta-Xylene | <0.5 | 25 | 0.30 | YES | 1-3 | positive |
| B** | | | | 0.30 | YES | 1-3 | positive |
| C | | | | 0.35 | YES | 1-3 | positive |
| D | Toluene | <0.5 | 60 | 0.23 | YES | 1-3 | positive |
| E | | | | 0.40 | YES | 1-3 | positive |
| F | | | | 0.47 | YES | 1-3 | positive |
| G | | | | 0.40 | YES | 1-3 | positive |
| H | | | | 0.30 | YES | 1-3 | positive |
| I | Methyl isobutyl ketone | 20 | 75.5 | 0.42 | YES | 1-3 | positive |
| J | Methyl pentyl ketone | 5 | 45 | 0.35 | YES | 1-3 | positive |
| K | Methoxycyclopentane | 12.5 | 30.5 | 0.30 | YES | 1-3 | positive |
| L | (CPME) | | | 0.40 | YES | 1-3 | positive |
| M | | | | 0.43 | YES | 1-3 | positive |
| N | n-Butyl acetate | 7 | 59 | 0.30 | YES | 1-3 | positive |
| O | | | | 0.36 | YES | 1-3 | positive |
| P | Isobutyl acetate | 7 | 59 | 0.36 | YES | 1-3 | positive |
| Q | | | | 0.36 | YES | 1-3 | positive |
| R | | | | 0.40 | YES | 1-3 | positive |
| S | | | | 0.40 | YES | 1-3 | positive |
| T | | | | 0.37 | YES | 1-3 | positive |
| U | Diethyl carbonate | 19 | 85 | 0.47 | YES | 1-3 | positive |

*the solidity after drying was evaluated as follows:
positive: the dried granule does not crush under light pressure from the spatula;
negative: the dried granule crushes under light pressure from the spatula and produces powder.

Test B carried out with a 5% aqueous HCl solution instead of water.

Following granulation as according to the invention, granules of size between 1 and 3 mm are obtained, which are sufficiently solid to be recovered, dried and handled.

### Example 2: Granulation in 2 L Reactor

### 1-Granulation and Drying:

### Granulation:

Equipment similar to that of Example 1 is used, but with a two-litre reactor. The quantities involved are given in the table below.

The stirrer is a stirrer with inclined blades and the initial stirring is set at 800 rpm, then lowered to 600 rpm after introduction of the organic binder as in the previous example.

### Drying of the Granules:

A porous glass filter with a diameter of 6 cm is used, fitted with a double jacket allowing the walls of the filter to be heated by circulating hot water. 100 g of undried filtered granules are introduced into the filter. A constant flow of nitrogen (2 L/min) is then injected through the bottom of the filter at different temperatures.

The granules are obtained as given in the table:

TABLE 3

| | | | Binder/ | | Size of |
|---|---|---|---|---|---|
| | | | AZDN | Formation | granules |
| | Water | AZDN | mass | of | obtained |
| Organic binder | (g) | (g) | ratio | granules | (mm) |
| meta-Xylene | 900 | 100 | 0.30 | YES | 2-4 |
| | 900* | 100 | 0.30 | YES | 2-4 |
| Methoxycy- | 900 | 100 | 0.45 | YES | 2-4 |
| clopentane | | | | | |
| (CPME) | | | | | |
| n-Butyl acetate | 900 | 100 | 0.36 | YES | 2-4 |
| Isobutyl acetate | 900 | 100 | 0.40 | YES | 2-4 |
| | 900 | 109** | 0.40 | YES | 2-4 |

*10% HCl aqueous solution
**starting from 109 g of wet AZDN containing 8.2% water, equivalent to 100 g of dry AZDN

### 2-Determination of the Residual Organic Binder Content:

During the drying of the granules described above, approximately one gram of granules obtained with isobutyl acetate is taken over time and analysed by gas chromatography to determine the residual binder content.

The results obtained are presented in the following table:

TABLE 4

Residual binder
content, % by mass

| t(h) | 40° C. | 30° C. |
|---|---|---|
| 0 | 22 | 22.22 |
| 1 | 11.65 | 15.17 |
| 2 | 1.59 | 7.80 |
| 3 | 0.02 | 0.30 |
| 6 | 0.02 | 0.02 |
| 9 | 0.02 | 0.01 |

With CMPE, at 30° C., drying is faster and after 3 hours a residual CPME concentration of 0.01% is observed, which does not change significantly thereafter (5 h drying).

The preparation and drying of the granules can therefore be carried out under satisfactory industrial conditions. After a few hours of drying, granules are obtained with a residual organic binder content by mass of between 0.01% and 0.02%, i.e. between 100 ppm and 200 ppm of residual organic binder (i.e. between 100 and 200 mg of organic binder per kg of granules).

### 3-Crushing Test on Dried Granules:

20 g of AZDN granules obtained in Example 2 are placed and distributed uniformly in a glass crystallizer 6 cm in diameter, then a flat-bottomed glass beaker 5 cm in diameter (with an empty mass of 200 g) is placed on top. If no crushed or broken granules are observed, additional weights of 200 g are gradually added above the beaker until the beginning of crushing of the granules is observed, visible through transparency in the bottom of the beaker or on the sides of the glass crystallizer.

The surface area of the beaker is S=π×(2.5)²=19.625 cm².

The crushing resistance is: [total weight (beaker+additional weight(s)) at breaking point]/[S]

### Comparative Test:

Granules were prepared by extrusion according to the procedure described in application WO 00/24706 in Examples 1 and 2. The AZDN powder is that used for the previous examples of the present application. The granules obtained have a diameter of 5 mm and a length of 1.5 cm on average (between 1 and 2 cm). Once dried, these granules appear very fragile when handled. They show very low resistance to the crushing test, much lower than that obtained for the granules according to the invention.

The results obtained are presented in the following table:

TABLE 5

| | Total mass | |
|---|---|---|
| | applied at | Crushing |
| | breaking | resistance |
| Organic binder | point (g) | (g/cm²) |
| m-Xylene | >=1200 | 61.15 |
| Isobutyl acetate | >=1200 | 61.15 |
| n-Butyl acetate | | |
| CPME | >=1200 | 61.15 |
| Comparative test as in WO 00/24706: | | |
| Granules without surfactant - Example 1 | 200 | 10.19 |
| Granules with surfactant* - Example 2 | 400 | 20.38 |

*with a C13-C15 fatty alcohol condensed with ethylene oxide and propylene oxide

The granules according to the invention have a significantly improved crushing resistance. They are easily handled without falling apart.

### Example 3: Granulation in a 100 L Reactor

### 1-Granulation and Drying:

A 100 litre AE100 reactor in enamelled stainless steel is used, of the DE DIETRICH brand. The internal diameter of the reactor vessel is 508 mm, the useful height is 375 mm.

Stirring is ensured by a 300 mm diameter “impeller”-type stirrer with three straight blades ensuring radial-type stirring. The stirring speed can vary from 0 to 200 revolutions per minute. The reactor is equipped with a ball-type bottom valve connected to a filter dryer. The filter diameter is 55 cm. The filter is equipped with a scraper agitator with a diameter of 48 cm which can go up or down in the filter and which, in the lowered position flush with the filter, allows mechanical evacuation of the dried product through a side opening. The stirring speed can vary from 0 to 60 rpm. The filtration cloth is a 20 micron mesh cloth.

The test is carried out at room temperature (19-20° C.).

The reactor is maintained under nitrogen by a light flush of 50 l/h.

43.1 kilograms of demineralized water are introduced. Stirring is set at 200 revolutions per minute, the temperature is 18° C. 11.3 kilograms of wet AZDN powder with 8% water content are introduced over 5 minutes.

After 5 min, 4.1 kilograms of isobutyl acetate are added over two minutes. The formation of granules is visible within the first few minutes. After 5 minutes, the stirring is lowered to 100 rpm and these conditions are maintained for 5 h.

The reactor is then drained through the bottom valve into the filter. The granules are filtered by applying nitrogen pressure in order to evacuate and recover the aqueous liquors.

A 10 m³/h nitrogen flush is then put in place for 24 h at room temperature.

The granules, once dried, are solid and can be recovered by starting the mechanical scraper agitator, set at a speed of 6 revolutions per minute. In this way, 10.2 kilograms of AZDN are recovered in the form of granules approximately 2 to 3 millimetres in diameter.

### 2-Crushing Resistance Test:

A crushing resistance test on the granules as described in the previous example was carried out and shows very good solidity of these granules with a resistance of up to 91.72 g/cm².

### 3-Dissolution Test:

A granule dissolution test is carried out at room temperature. A glass Erlenmeyer flask fitted with a magnetic stirring bar is placed on a magnetic stirrer. With the stirring stopped, 13 g of AZDN are introduced into the Erlenmeyer flask. 100 ml of acetone are then introduced quickly and stirring is started (100 rpm). The time taken until, visually, undissolved AZDN crystals are no longer seen is recorded.

Commercial recrystallized AZDN (130-257-438 microns/dv 10-50-90), AZDN granules prepared and dried as obtained above, and the dry powdered AZDN used in Example 1 are compared.

TABLE 6

| | | Spherical AZDN | Recrystallized |
|---|---|---|---|
| | Dry AZDN powder | (granules 2-3 mm) | AZDN |
| | (Dv 50 = 100 | According to | (Dv 50 = |
| | microns) | the invention | 247 microns) |
| Dissolution | 15 | 15 | 35 |
| time | | | |
| (seconds) | | | |

It is seen that the spherical AZDN granulates obtained according to the invention dissolve as quickly as the AZDN in initial powder form. On the other hand, the recrystallized AZDN crystals, although having an average diameter less than that of the spherical granules of the invention, take much longer to dissolve in acetone.

No visible insoluble matter is recorded for each of the samples.

1. Process for granulating an azo compound of general formula (I) below: 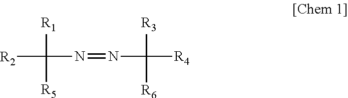
in which:
the radicals R¹, R², R³ and R⁴, identical or different, are chosen independently of one another from:
a linear or branched alkyl group, optionally substituted by a hydroxy or alkoxy group or by a halogen atom;
a cycloalkyl group, optionally substituted by a hydroxy or alkoxy group or by a halogen atom;
an aryl group, optionally substituted by a hydroxy, alkyl or alkoxy group or by a halogen atom;
an aralkyl group, optionally substituted by one or more alkyl, alkoxy or hydroxy group(s) or by one or more halogen atom(s); or
at least one of the combinations of R¹ with R² and/or of R³ with R⁴ forms, with the carbon atom to which it is (or they are) connected, a cycloalkyl radical or a C(O) group;
the radicals R⁵ and R⁶, identical or different, are chosen independently of one another from the CN or NH₂ groups;
said process comprising the following steps:
a) a granulation step by stirring an aqueous suspension of said azo compound, in the presence of an organic binder;
b) an optional step of recovering the granules obtained in step a), preferably by filtration; and
c) an optional step of drying the granules recovered in step b).

2. Granulation process according to claim 1, in which said organic binder is added to said aqueous suspension.

3. Granulation process according to claim 1, in which the azo compound of general formula (I) is chosen from the group consisting of:
2,2′-azobis(isobutyronitrile), 2,2′-azobis(2,4-dimethylvaleronitrile), 2,2′-azobis(2-methylhexylonitrile), 2,2′-azobis(2-cyclopropylpropionitrile), 2,2′-azobis(2-phenylpropionitrile), 2,2′-azobis(2-methylbutyronitrile), 1, l′-azobis(1-cyclohexanecarbonitrile) and azodicarbonamide; preferably 2,2′-azobis(isobutyronitrile).

4. Granulation process according to claim 1, in which the organic binder is chosen from aliphatic acetates, aliphatic carbonates, non-halogenated aromatic hydrocarbons, aliphatic ketones and aliphatic ethers.

5. Granulation process according to claim 1, in which the azo compound/water mass ratio can be between 1/99 and 40/60, preferably between 10/90 and 25/75; and more preferably between 15/85 to 25/75.

6. Granulation process according to claim 1, in which the organic binder/azo compound mass ratio can be between 0.2 and 0.5, preferably between 0.3 and 0.5, more preferably between 0.3 and 0.4.

7. Granules comprising an azo compound of general formula (I) and an organic binder as defined in claim 1.

8. Granules according to claim 7, characterized in that they are of substantially spherical shape, preferably with a diameter of between 0.5 and 5 mm.

9. Granules obtainable by the process such as according to claim 1.

10. Use of the granules according to claim 7, as swelling agents, initiators of polymerization reactions using free radicals, or else as synthesis intermediates, particularly in the preparation of pharmaceutical or agrochemical compounds.
